(19) United States
(12) Patent Application Publication
(10) Pub. No.: US 2020/0138397 A1
(43) Pub. Date: May 7, 2020

(54) OPERATION MANAGEMENT SYSTEM, MOBILE RADIATION GENERATING DEVICE, AND MOBILE RADIOGRAPHIC IMAGING SYSTEM

(71) Applicant: Konica Minolta, Inc., Tokyo (JP)

(72) Inventors: Masaki SUZUKI, Tokyo (JP); Hajime ISHIMOTO, Tokyo (JP); Takafumi MATSUO, Tokyo (JP)

(21) Appl. No.: 16/674,277

(22) Filed: Nov. 5, 2019

(30) Foreign Application Priority Data
Nov. 7, 2018 (JP) 2018-209316

(51) Int. Cl. A61B 6/00

(52) U.S. Cl. CPC A61B 6/548; A61B 6/461; A61B 6/40

(57) ABSTRACT

An operation management system for managing a mobile radiation generating device includes a hardware processor that repeatedly acquires positional information including a position at which the mobile radiation generating device is present and a time when the mobile radiation generating device is present at the position, sequentially records positional information as repeatedly acquired in a memory, generates operation information related to a past operation of the mobile radiation generating device on the basis of a plurality of pieces of positional information as recorded, and causes a display to display the operation information as generated.

### CROSS REFERENCE TO RELATED APPLICATION

The present invention claims priority under 35 U.S.C. § 119 to Japanese Patent Application No. 2018-209316, filed on Nov. 7, 2018, the entire content of which is incorporated herein by reference.

### BACKGROUND

### 1. Technological Field

The present invention relates to an operation management system, a mobile radiation generating device, and a mobile radiographic imaging system.

### 2. Description of the Related Art

Conventionally, it is common to capture a radiographic image in an imaging room in which a radiation generating device that generates radiation and an imaging table in which a radiation detecting device that generates a radiographic image in accordance with received radiation is loaded are installed respectively, but in recent years, radiation generating devices are being provided with mobility with a moving device such as a wheel in order to facilitate imaging a subject difficult to move.

In addition, for the purpose of smoothly operating such mobile radiation generating device and radiation detecting device, various management systems for managing them are being developed.

As an example of such systems, an imaging information management system is described in JP2006-340788A, for example, which includes a plurality of mobile X-ray imaging devices that accept input of round status information indicating a round status for each round order of a round scheduled patient and transmit the input round status information, and a centralized management device that accepts input of round order information and round scheduled time information, receives round status information from the mobile X-ray imaging devices, and displays the input round order information and round scheduled time information in comparison with the received round status information.

In addition, a round-purpose radiographic imaging device management system is described in JP2008-000430A which includes a round-purpose X-ray imaging device that holds its own position and orientation to be used as a reference, counts the number of revolutions of wheels following the movement from the held position, calculates its own position after the movement using the held position and orientation as well as the counted number of revolutions of wheels, and transmits the position as positional information, and a centralized management device that receives the positional information from the round-purpose X-ray imaging device and displays the position of the round-purpose X-ray imaging device on the basis of the positional information.

However, the information that the imaging information management system described in JP2006-340788A displays is a comparison between an original proceeding schedule and the current status of progress, and the information is not left after the operation.

In addition, the information that the round-purpose radiographic imaging device management system described in JP2008-000430A displays is also the current positional information, and the information is not left after the operation.

That is, conventional management systems as those described in JP2006-340788A and JP2008-000430A are intended to inform a user of the current status of a mobile radiographic imaging device being operated, so that the user can take an action only on a case-by-case basis (such as, for example, adjusting an imaging duration in a round currently being performed) even he/she sees the displayed information. That is, it is difficult with information that conventional management systems display to resolve operational problems of mobile radiographic imaging devices having caused such an action to be taken.

### SUMMARY

The present invention has an object to easily increase the efficiency of future operations of a mobile radiation generating device having a radiation generator capable of generating radiation and being configured to be movable.

To achieve at least one of the abovementioned objects, according to a first aspect of the present invention, an operation management system for managing a mobile radiation generating device has a radiation generator capable of generating radiation, the mobile radiation generating device being configured to be movable, the operation management system comprising:

a hardware processor that

repeatedly acquires positional information including a position at which the mobile radiation generating device is present and a time when the mobile radiation generating device is present at the position,

sequentially records the positional information as repeatedly acquired in a memory,

generates operation information related to a past operation of the mobile radiation generating device on a basis of a plurality of pieces of positional information as recorded, and

causes a display to display the operation information as generated.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 is a side view of a mobile radiographic imaging system according to a first embodiment of the present invention.

FIG. 2 is a block diagram representing an operation management system included in the mobile radiographic imaging system shown in FIG. 1.

FIG. 3 is a plan view of the inside of a facility in which the mobile radiographic imaging system shown in FIG. 1 is included.

FIG. 4 is a plan view of the inside of the facility in which the mobile radiographic imaging system shown in FIG. 1 is included.

FIG. 5 shows an exemplary display screen that the operation management system shown in FIG. 2 displays.

FIG. 6 shows an exemplary display screen that the operation management system shown in FIG. 2 displays.

FIG. 7 shows an exemplary display screen that the operation management system shown in FIG. 2 displays.

FIG. 8 shows an exemplary display screen that the operation management system shown in FIG. 2 displays.

FIG. 9 shows an exemplary display screen that the operation management system shown in FIG. 2 displays.

FIG. 10 shows an exemplary display screen that the operation management system shown in FIG. 2 displays.

FIG. 11 shows an exemplary display screen that the operation management system shown in FIG. 2 displays.

FIG. 12 is a side view of a mobile radiation generating device and a radiation detecting device according to a second embodiment of the present invention.

FIG. 13 is a block diagram representing a radiation generator of the mobile radiation generating device shown in FIG. 12.

FIG. 14A and FIG. 14B are tables showing various types of information recorded by the mobile radiographic imaging system shown in FIG. 1 or the mobile radiation generating device shown in FIG. 12.

### DETAILED DESCRIPTION OF EMBODIMENTS

Hereinafter, one or more embodiment of the present invention will be described with reference to the drawings. However, the scope of the invention is not limited to the disclosed embodiments.

### First Embodiment

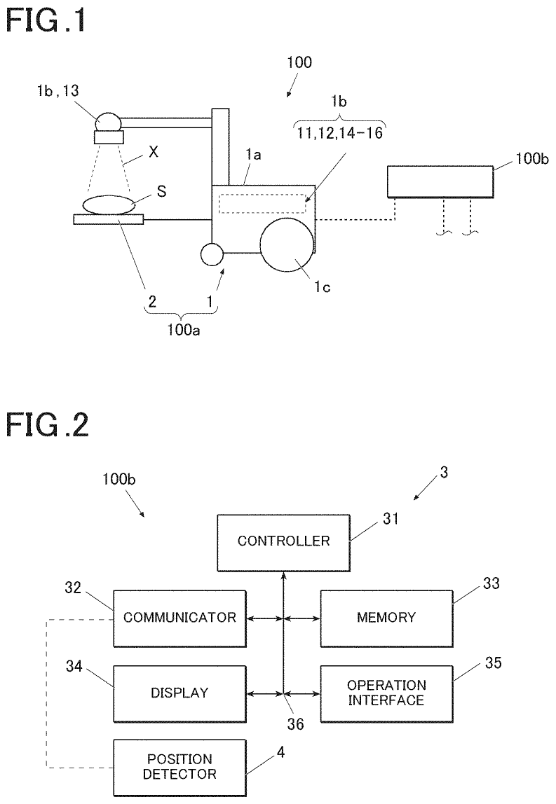

A first embodiment of the present invention will be described first with reference to FIG. 1 to FIG. 11.

Mobile Radiographic Imaging System

First, a schematic configuration of a mobile radiographic imaging system (hereinafter, an imaging system 100) according to the present embodiment will be described. FIG. 1 is a side view of the imaging system 100.

The imaging system 100 includes a mobile radiographic imaging device (hereinafter, an imaging device 100a) and an operation management system 100b, as shown in FIG. 1.

Note that a single imaging device 100a or a plurality of imaging devices 100a may be provided for a single operation management system 100b.

In addition, the imaging system 100 may be connectable to another system such as a radiology information system (RIS) not shown or a picture archiving and communication system (PACS).

The imaging device 100a includes a mobile radiation generating device (hereinafter, a medication cart 1) and a radiation detecting device 2.

Note that the imaging device 100a may only capture still images, or may be adaptable to video capturing.

Note that the imaging device 100a may include a console with which imaging conditions and the like can be set. In that case, the console may be mounted on the medication cart 1.

The medication cart 1 has an enclosure 1a, a radiation generator 1b, and a wheel 1c.

The radiation generator 1b includes a high voltage generator 12 that, on the basis of the fact that an irradiation instructing switch 11 is operated, generates a voltage in accordance with an imaging condition set in advance, a radiation source 13 that, when the voltage is applied from the high voltage generator 12, generates a dose of radiation (for example, X-ray or the like) in accordance with the applied voltage, a controller 14 that controls the high voltage generator, a memory 15 in which various programs that causes the controller to operate are held, a communicator 16 for transmitting/receiving various control signals, various types of data, and the like to/from another device (for example, the radiation detecting device 2), and the like.

Note that the medication cart 1 may be of a hand-propelled type, or may include a power source such as a motor.

In addition, the medication cart 1 may include a moving device other than the wheel 1c, or may be reduced in size and weight so as to become simply portable without including a moving device including the wheel 1c.

In addition, the medication cart 1 may be configured to include an internal battery to receive supply of electric power from the battery, or may be configured to be capable of receiving supply of electric power from an outlet provided at an imaging place.

The radiation detecting device 2 is configured to be capable of generating a radiographic image of the subject in accordance with received radiation.

Specifically, the radiation detecting device 2 includes a radiation detector in which pixels including a radiation detection element that receives radiation to generate charges in accordance with a dose and a switching element are arrayed two-dimensionally (as a matrix), a reader that reads out the amount of charges discharged from each of the pixels as a signal value, and generates data on a radiographic image from a plurality of signal values, a controller that controls the radiation detecting device 2, a memory in which various programs that causes the controller to operate are held, a communicator for transmitting/receiving various control signals, various types of data, and the like to/from another device (for example, the medication cart 1) and transmitting the generated data on a radiographic image to another device, and the like.

Note that the radiation detecting device 2 may be of a type (what is called an indirect type) that internally includes a scintillator or the like, converts irradiated radiation to light of another wavelength, such as visible light, with the scintillator, and generates charges in accordance with the converted light, or may be of a type (what is called a direct type) that directly generates charges from radiation without the intervention of a scintillator or the like.

In addition, the radiation detecting device 2 may be of a cooperation system that performs the above-described imaging operation on the basis of a signal or the like received from another device, or may be of a non-cooperation system that is triggered by sensing of radiation from the medication cart 1 to automatically proceed into imaging.

In addition, the radiation detecting device 2 may be a film, or a cassette in which a fluorescent screen for use in computed radiography (CR) is stored.

By arranging the radiation source 13 of the medication cart 1 and the radiation detecting device 2 configured in this manner oppositely, arranging a subject S (human, animal, object) between them, and irradiating the subject S and the radiation detecting device 2 with radiation X from the radiation source 13, the radiation detecting device 2 generates a radiographic image of the subject.

Operation Management System

Next, a specific configuration of the operation management system 100b included in the above-described imaging system 100 will be described. FIG. 2 is a block diagram representing the operation management system 100b.

The operation management system 100b includes a system body 3 and a position detector 4 as shown in FIG. 2, and is configured to be capable of managing the medication cart 1.

Note that a management target of the operation management system 100b may be the detection device 2, or may be the imaging device 100a (the medication cart 1 and the detection device 2).

Hereinafter, a case where the imaging device 100a is a management target will be described.

The system body 3 includes a controller 31, a communicator 32, a memory 33, a display 34, an operation interface 35, and a bus 36 that connects them.

The controller 31 is composed of a central processing unit (CPU), a random access memory (RAM), and the like. The CPU of the controller 31 reads out various programs held in the memory 33 in accordance with an operation of the operation interface 35 for expansion into the RAM, and executes various types of processing in accordance with the expanded programs to exert centralized control over the respective components of the system body 3.

The communicator 32 is composed of a network interface and the like, and is capable of transmitting/receiving data to/from another device connected in a wired or wireless manner via a communication network such as LAN, WAN, or the Internet.

The memory 33 is composed of a nonvolatile semiconductor memory, a hard disk, or the like, and holds various programs to be executed by the controller 31, parameters necessary for execution of processing by the programs, and the like.

The display 34 is composed of a monitor of a liquid crystal display (LCD), a cathode ray tube (CRT), or the like, and is capable of displaying an instruction input via the operation interface 35, a radiographic image, and the like in accordance with an instruction of a display signal input from the controller 31.

The operation interface 35 includes a keyboard including a cursor key, number input keys, various functional keys, and the like as well as a pointing device such as a mouse, and outputs an instruction signal input through a key operation on the keyboard or a mouse operation to the controller 31.

In addition, the operation interface 35 may include a touch panel on a display screen of the display 34, and in this case, outputs an instruction signal input via the touch panel to the controller 31.

The position detector 4 is composed of a receiver of a global positioning system (GPS) included in the medication cart 1, an access point (AP) which is provided within a facility 200 and relays wireless communication (for example, Wi-fi) between the medication cart 1 and the system body 3, or the like, for example, and is configured to be capable of repeatedly detecting the position at which the imaging device 100a is present.

In a case where a GPS receiver serves as the position detector 4, a position calculated on the basis of radio waves that the receiver has received from a GPS satellite is transmitted to the system body 3. On the other hand, in a case where an AP serves as the position detector 4, a position calculated by the AP on the basis of the strength of radio waves arrived from the imaging device 100a (the medication cart 1 or the radiation detecting device 2) is transmitted to the system body 3.

In a case where the position detector 4 is mounted on the medication cart 1, the medication cart 1 can be specified easily. On the other hand, in a case of a building having a plurality of floors, it is difficult to identify the difference between the floors with the GPS; however, if an AP is used, it can be dealt with by installing an AP on each floor.

Note that the medication cart 1 may be configured to include a rotation measurer that measures the rotation of the wheel 1c and a height measurer that measures the height at which the medication cart 1 is located, so that the controller of the medication cart calculates the position on the basis of measurement results obtained by these measurers.

Alternatively, rather than calculating the position with a GPS receiver, an AP, or the medication cart, information included in radio waves received from a GPS satellite, the strength of arrived radio waves, and measurement results obtained by the measurers may be directly transmitted to the system body 3 for calculation in the controller 31 of the system body 3.

Alternatively, a monitoring camera, a laser radar, or the like provided within the facility 200 may serve as the position detector 4. In a case where a monitoring camera serves as the position detector 4, the medication cart 1 appearing in a captured image is analyzed by the controller 31 of the system body 3 to calculate the distance from the monitoring camera to the medication cart 1.

On the other hand, in a case where a laser radar serves as the position detector 4, the medication cart 1 is irradiated with laser light, and the distance from the laser radar to the medication cart 1 is calculated on the basis of reflected light.

Although a device that identifies a moving object is required in a case where the position detector 4 is configured in this manner, transmission of radio waves or the like from the medication cart 1 is unnecessary. In addition, it is not necessary to provide the position detector 4 for each medication cart 1, so that a case of introducing a new medication cart can be dealt with immediately, and the position of the medication cart 1 can be specified easily without facility information.

The controller 31 of the operation management system 100b according to the present embodiment configured in this manner has the function of repeatedly acquiring positional information.

Here, the “positional information” includes the position at which the imaging device 100a is present (the position calculated and transmitted by the position detector 4) and the time when the imaging device 100a is present at the position.

Specifically, the positional information output from the position detector 4 is received via the communicator 32.

In addition, the controller 31 in the present embodiment further acquires at least any information of imaging information, operator information, subject information, and facility information, in addition to the positional information.

Here, the “imaging information” is information related to details of imaging through use of the imaging device 100a, and specifically refers to an imaging area, imaging direction, imaging condition, imaging place, and the like.

In addition, the “operator information” is information related to an operator who operates the imaging device 100a, and specifically refers to the operator's name, ID, and the like.

In addition, the “subject information” is information related to a subject to be an imaging target in imaging through use of the imaging device 100a, and specifically refers to the subject's name, ID, disease name, and the like.

In addition, the “facility information” is information related to the facility 200 in which the imaging device 100a is included, and specifically includes a plan view Pv of the inside of the facility 200.

Note that branch point information, waiting place information, and imaging place information are included in the facility information in the present embodiment.

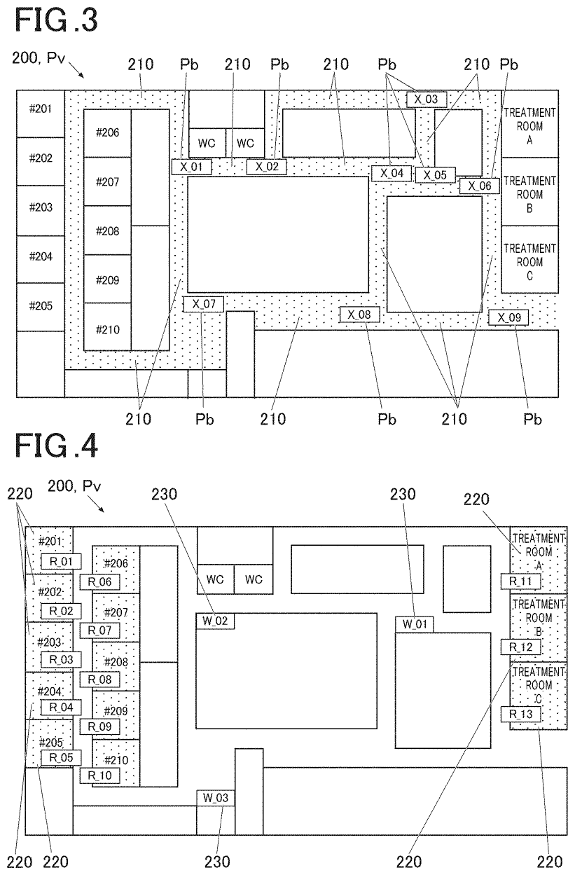

Here, the “branch point information” is information related to a branch point Pb on a passage 210 in the facility 200 along which the imaging device 100a can move, and specifically refers to the position (coordinates or the like) at which the branch point Pb exists. Note that, in a case where a plurality of branch points Pb exist, the ID that identifies each of the branch points Pb and the like are also included in the branch point information. For example, in a case where the facility 200 is represented by the plan view Pv as shown in FIG. 3, pieces of branch point information corresponding to the branch points Pb at a plurality of positions (nine positions) are acquired respectively, and the respective pieces of branch point information are provided with IDs (for example, X_01 to X_09).

In addition, the “imaging place information” is information related to a place at which imaging is performed, and specifically refers to the position (coordinates or the like) at which a room 220 in which imaging is performed exists. In a case where a plurality of imaging places exist, the ID that identifies each of the imaging places and the like are also included in the imaging place information. For example, in a case where the facility 200 is represented by the plan view Pv as shown in FIG. 4, pieces of imaging place information corresponding to the rooms 220 at a plurality of positions (thirteen positions) are acquired respectively, and the respective pieces of imaging place information are provided with IDs (R_01 to R_13).

In addition, the “waiting place information” is information related to a waiting place 230 for the imaging device 100a when not being used, and specifically refers to the position (coordinates or the like) at which the waiting place 230 exists. Note that, in a case where a plurality of waiting places exist, the ID that identifies each of the waiting places 230 and the like are also included in the waiting place information. For example, in the case where the facility 200 is represented by the plan view Pv as shown in FIG. 4, pieces of waiting place information corresponding to the waiting places 230 at a plurality of positions (three positions) are acquired respectively, and the respective pieces of waiting place information are provided with IDs (W_01 to W_03).

In addition, the controller 31 has the function of sequentially recording various types of information including repeatedly acquired positional information. In the present embodiment, various types of information are accumulated in a database provided in the memory 33.

Note that various types of information may be accumulated in an external server rather than in the memory 33.

In addition, the controller 31 has the function of generating operation information on the basis of a plurality of pieces of recorded positional information.

Here, the “operation information” is information related to past operations of the imaging device 100a, and specifically refers to a movement path of the imaging device 100a, a movement duration required for movement, an imaging duration required for imaging, and the like.

In addition, the controller 31 has the function of displaying generated operation information. In the present embodiment, the display 34 is caused to display the generated operation information.

Note that the operation information may be displayed following the fact that a user designates a next imaging place, or may be displayed automatically on the basis of imaging order information.

The controller 31 in the present embodiment generates operation information on the basis of further acquired information among imaging information, operator information, subject information, and facility information, in addition to a plurality of pieces of positional information.

For example, the controller 31 generates path information on the basis of a plurality of pieces of recorded positional information and facility information.

Here, the “path information” is information related to a movement path when the imaging device 100a moves within the facility 200. The path information can be a set of respective pieces of positional information, for example, or can be a combination of pieces of information about the waiting place 230 as a starting point, a travel direction when passing through the branch point Pb, and the room 220 as a goal.

Then, the controller 31 is capable of causing the display 34 to display the plan view Pv of the inside of the facility 200 on the basis of the facility information, and causing a movement path R of the imaging device 100a to be displayed in a superimposed manner on the plan view Pv on the basis of the generated path information.

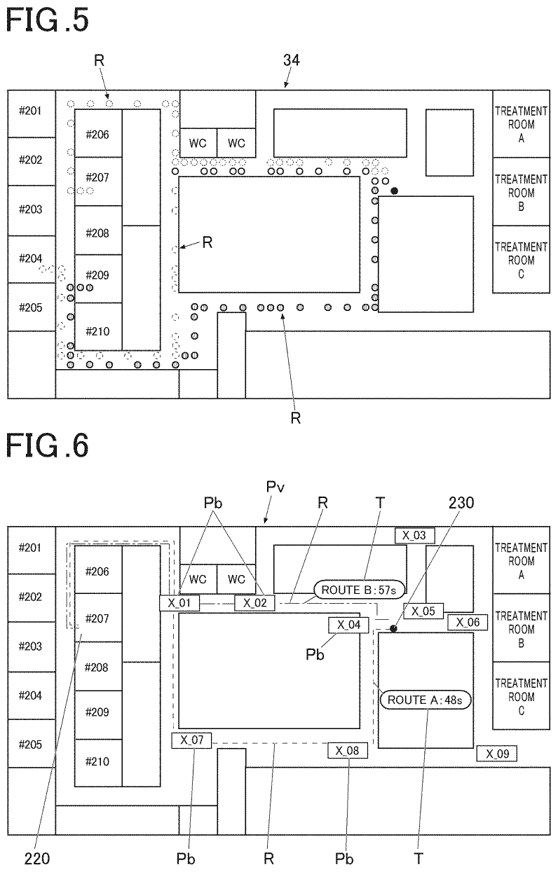

Specifically, as shown in FIG. 5, for example, the path information may be displayed as a collection of points plotted at positions indicated by individual pieces of positional information, or may be displayed as a line that connects coordinates indicated by individual pieces of positional information. Alternatively, as shown in FIG. 6, for example, the path information may be displayed as a line that connects the waiting place 230 confirmed by positional information as being a starting point, each of the branch points Pb confirmed that the imaging device 100a has passed through, and an imaging place (the room 220) confirmed as being an end point.

Note that, when displaying the movement path, it is preferable to cause an indication T of a movement duration taken for the imaging device 100a to move along the movement path to be displayed in the vicinity of the movement path, as shown in FIG. 6.

In addition, in a case where a plurality of pieces of path information have been recorded, a plurality of movement paths are displayed. In that case, it is preferable to change the display mode of each of the movement paths, as shown in FIG. 6. Although the movement paths are distinguished by different line types in FIG. 6, the color, line thickness, or the like may be changed.

Accordingly, since a movement path having achieved the earliest arrival at an imaging place in the past can be recognized at a glance, it is possible to cause the imaging device 100a to move for a short while by selecting that movement path, leading to a reduction of burdens for a user and an increase in efficiency of business operation for a facility manager.

In addition, a reduction of user's work can achieve appropriate labor management.

Note that, when generating path information, various types of information to be used may be filtered by items such as each medication cart, a user, a movement speed, an imaging duration, imaging information, an imaging date and time, and a facility (in a case of managing a plurality of facilities), so that the movement path R to be displayed can be changed for each feature quantity.

In addition, the controller 31 in the present embodiment divides the generated path information using the branch points Pb indicated by the branch point information as boundaries to generate respective pieces of partial path information related to a plurality of partial paths Rs corresponding to the respective sections 211 obtained by separating the passage 210 at the branch points Pb.

In addition, the controller 31 in the present embodiment generates section data including a movement duration that the imaging device 100a has required to move through the corresponding sections 211 on the basis of the generated partial path information.

Note that, in a case where a plurality of pieces of partial path information corresponding to an identical section 211 exist, an average value of respective movement durations may be calculated on the basis of the plurality of pieces of partial path information, and section data including the average value may be generated.

In addition, by previously acquiring the distance of the respective sections 211, the movement speed when moving through the respective sections 211 may be calculated, and may be included in the section data.

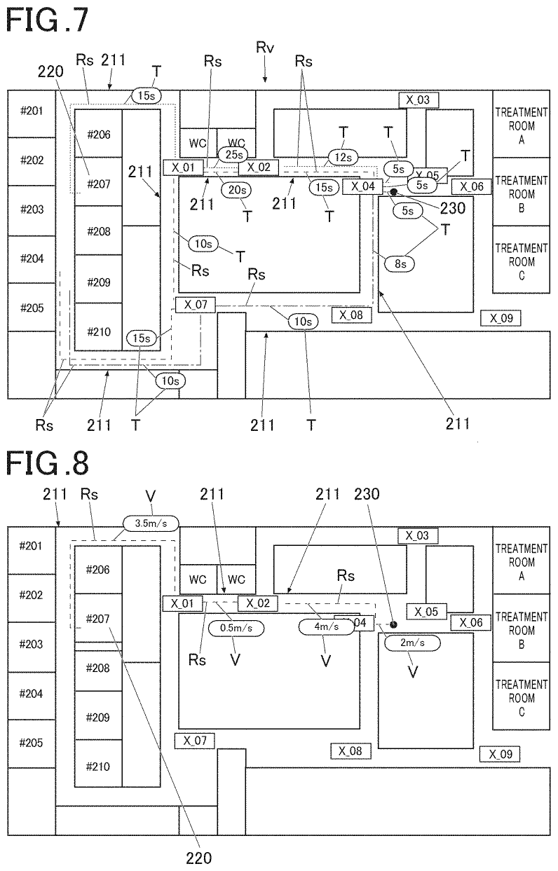

Then, the controller 31 is capable of causing the display 34 to display the plan view Pv of the inside of the facility 200 and the movement path R on the basis of facility information and path information, and as shown in FIG. 7, for example, causing the indications T of the movement duration in the respective sections 211 to be displayed in the vicinity of the corresponding partial paths Rs on the basis of the generated section data.

Note that, when calculating the movement speed, it may be configured such that the indication T of the movement duration in each of the sections 211 can be switched to an indication V of the movement speed, as shown in FIG. 8, for example.

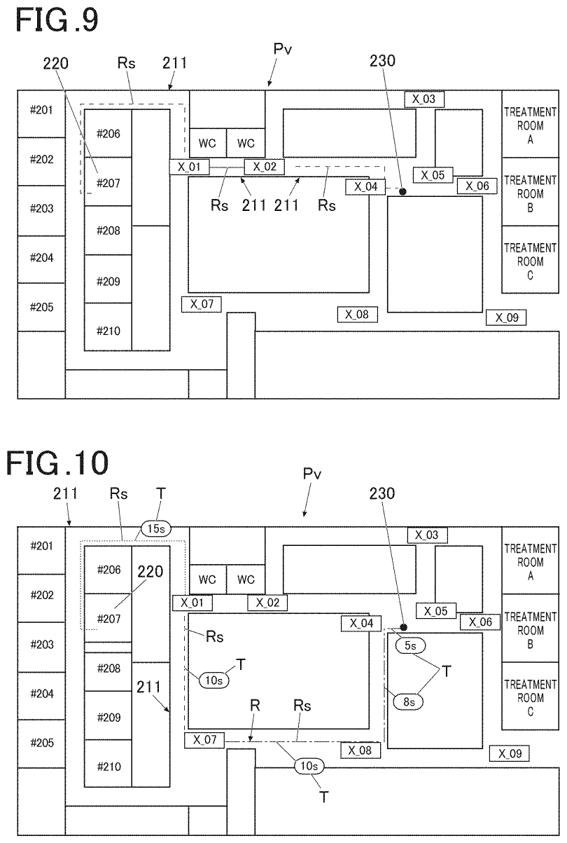

On that occasion, the controller 31 may cause the display mode of the corresponding partial paths Rs to be changed in accordance with the extent of the movement duration included in the section data. Specifically, as shown in FIG. 9, for example, the color, line type, line thickness, or the like of the partial path Rs (in FIG. 9, between the branch point Pb depicted as “X_01” and the branch point Pb depicted as “X_02”) in which the movement speed is slow is changed.

Accordingly, the section 211 in which a long time was taken to move through in the past can be recognized at a glance, so that the reason why it takes time to move through that section 211 can be deduced by considering a situation around that section. For example, in a case where it takes time in the section 211 in front of restrooms as in FIG. 9, it can be deduced that many people are gathering in front of the restrooms and obstructing the passage. Thus, by causing the imaging device 100a to move avoiding this section 211, the imaging device 100a can be moved for a short while, leading to a reduction of burdens for a user and an increase in efficiency of business operation for a facility manager.

In addition, a reduction of user's work can achieve appropriate labor management.

In addition, the controller 31 in the present embodiment extracts partial path information corresponding to section data that achieve the shortest movement duration from a plurality of pieces of partial path information corresponding to an identical section 211 as shortest partial path information, and combines extracted pieces of shortest partial path information corresponding to the respective sections 211 to generate shortest path information related to a movement path that can achieve the shortest movement to an imaging place.

Then, the controller 31 is capable of causing the display 34 to display the plan view Pv of the inside of the facility 200 on the basis of facility information, and causing the shortest movement path R of the imaging device 100a to be displayed in a superimposed manner on the plan view Pv on the basis of the generated shortest path information as shown in FIG. 10.

On that occasion, the indications T of the movement durations of the entire movement path R or the respective partial paths Rs may be displayed together.

Accordingly, the movement path R that can achieve still faster movement than the movement path R determined in the past as achieving the shortest movement could be found. If such a movement path R can be newly found, it is possible to cause the imaging device 100a to move for a short while, leading to a reduction of burdens for a user and an increase in efficiency of business operation for a facility manager.

In addition, a reduction of user's work can achieve appropriate labor management.

In addition, in a case where there are a plurality of waiting places 230, the controller 31 in the present embodiment generates shortest waiting place information on the basis of the imaging information, waiting place information, and a plurality of pieces of section data.

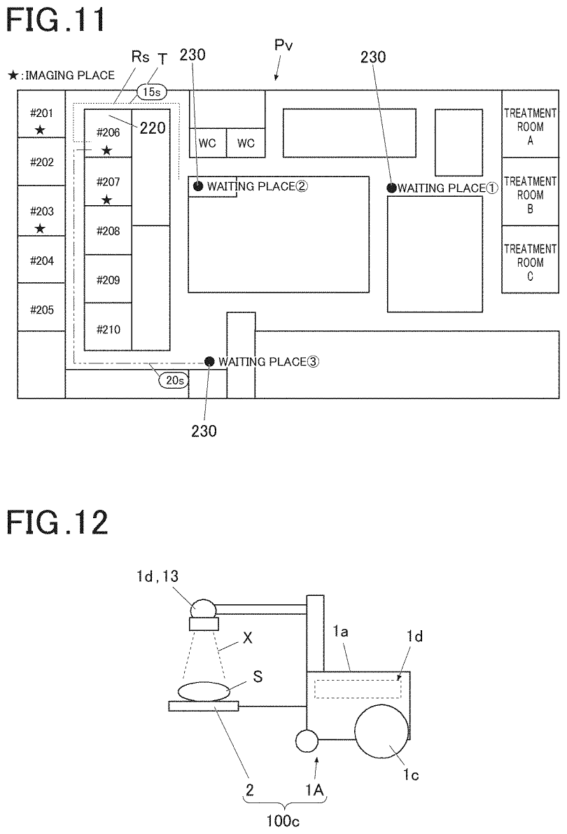

Here, the “shortest waiting place information” is information related to the waiting place 230 achieved the shortest movement duration to an imaging place at which imaging has been performed, and specifically refers to the position (coordinates or the like) at which the waiting place exists. As shown in FIG. 11, for example, the movement durations from the respective waiting places 230 to a predetermined imaging place (the room 220) are calculated respectively, and among them, the position at which the waiting place 230 having required the shortest duration exists is determined as the shortest waiting place information. In a case where a room 206 shown in FIG. 11 is determined as an imaging place, the waiting place 230 depicted as a “waiting place (2)” is the waiting place 230 having required the shortest duration.

Note that a plurality of pieces of section data to be used may be filtered by a time zone or day of the week, and then the shortest waiting place information may be generated.

Then, the controller 31 is capable of indicating the position of the waiting place 230 that can achieve the shortest movement to the imaging place on the plan view Pv on the basis of the shortest waiting place information.

Note that, as to the manner of indicating the waiting place 230, only the waiting place 230 that can achieve the shortest movement may be displayed, or only the waiting place 230 that can achieve the shortest movement may be displayed in a specific display mode while displaying all the waiting places 230.

On that occasion, the movement path R from the waiting place 230 that can achieve the shortest movement to the imaging place may be displayed together.

Accordingly, by causing the imaging device 100a to wait at an indicated waiting place after a previous operation, it is possible to cause the imaging device 100a to move to an initial imaging place for a short while in the next operation, leading to a reduction of burdens for a user and an increase in efficiency of business operation for a facility manager.

In addition, a reduction of user's work can achieve appropriate labor management.

Note that a plurality of pieces of section data to be used may be filtered by a time zone or day of the week, and then the shortest waiting place information may be generated. Accordingly, a more correct waiting place 230 can be selected because the imaging place and ease of passing along a certain passage (the degree of crowding or the like) may vary depending on the time or day of the week.

In addition, in a case of acquiring imaging information, the controller 31 in the present embodiment generates an imaging duration required for imaging on the basis of a plurality of pieces of positional information and imaging information as acquired.

Specifically, for example, a duration after it is detected that the imaging device 100a has stopped at an imaging place indicated in imaging information and until it is detected that the imaging device 100a starts moving again is timed as an imaging duration, or a duration after a signal that notifies the start of imaging is received from the imaging device 100a upon receipt of positional information at the imaging place indicated in the imaging information and until a signal that notifies the end of imaging is received is timed.

Then, the controller 31 is capable of displaying imaging information, user's name, imaging duration, and the like on the display 34 as a list.

Accordingly, by comparing imaging durations in an identical type of imaging in the past, a user who takes relatively long time can be identified. The user who takes time may have poor skills, and measures such as clarifying skills to be improved and providing the user with efficient retraining can be taken. In addition, an imaging plan and a round route in conformity with characteristics of a technologist can also be set.

Further, a developer of the imaging device 100a can also understand the operation mode, and can provide feedback to development of a next model.

Note that only the position of the medication cart 1 may be recorded at certain time intervals, imaging information may be acquired when performing an analysis, and whether or not imaging was performed at that time may be displayed, or imaging information may be acquired in advance, and positional information at the start and end of imaging may be recorded.

Unlike conventional systems, the operation management system 100b according to the present embodiment described above records past positional information, and generates operation information related to past operations of the imaging device 100a on the basis of a plurality of pieces of recorded positional information. Thus, by referring to the operation information before operating the imaging device 100a at subsequent times, a user can devise an efficient imaging plan, and can improve labor management (personnel allocation), equipment management, and the like (eliminate a time-consuming factor from a time-consuming movement path), and formulate a training plan. Then, as a result, future operations of the imaging device 100a can be easily increased in efficiency.

### Second Embodiment

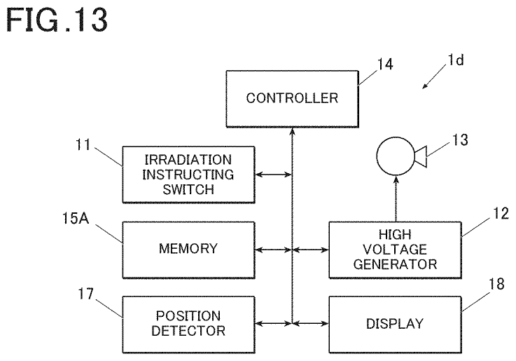

Next, a second embodiment of the present invention will be described with reference to FIG. 12 and FIG. 13. Note that components similar to those of the above-described first embodiment will be denoted here by identical reference characters, and description thereof will be omitted.

The invention according to the above-described first embodiment is the mobile radiographic imaging system including the imaging device 100a and the operation management system 100b, whilst an invention according to the present embodiment is a mobile radiographic imaging device (hereinafter, an imaging device 100c), and does not include a component equivalent to the operation management system 100b of the above-described embodiment.

Instead, the imaging device 100c has various functions in the above-described operation management system 100b.

Specifically, the imaging device 100c includes a mobile radiation generating device (hereinafter, a medication cart 1A) and the radiation detecting device 2 similar to that of the first embodiment, as shown in FIG. 12, for example.

In addition, the medication cart 1A includes a radiation generator 1d in addition to the enclosure 1a and the wheel 1c similar to those of the first embodiment.

The radiation generator 1d according to the present embodiment includes a memory 15A, a position detector 17, and a display 18 in addition to the irradiation instructing switch 11, the high voltage generator 12, the radiation source 13, and the controller 14 similar to those of the first embodiment, as shown in FIG. 13, for example.

In the present embodiment, the medication cart 1A acquires positional information by itself, and thus, a component equivalent to the communicator 16 in the first embodiment may not be provided.

In the memory 15A, a program for causing the medication cart 1A to function as the operation management system is also held in addition to the programs held in the memory 15 of the above-described first embodiment.

The position detector 17 is similar to the position detector 4 according to the above-described first embodiment.

With the imaging device 100c according to the present embodiment described above, future operations of the imaging device 100c (itself) can be easily increased in efficiency similarly to the above-described first embodiment.

Others

A facility manager wishes to increase the efficiency of future operations of the medication cart 1 (1A), and a supervisor of users wishes to visualize the difference in skill between the respective users. In addition, a service representative of a manufacturer of the imaging device 100a (100c) and the facility manager wish to make it easy to consider the frequency of maintenance of the medication cart 1 (1A), and a used equipment buying/selling company that handles the imaging device 100a (100c) wishes to easily assess the price of the medication cart 1 (1A) having been used. That is, parties relevant to the medication cart 1 (1A) always require information about the medication cart 1 (1A).

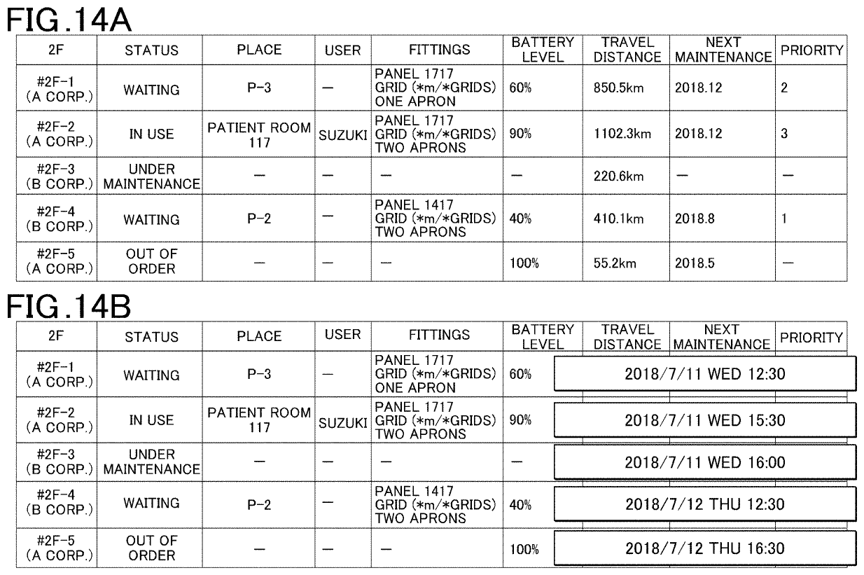

Thus, in the operation management system 100b according to the above-described first embodiment or the imaging device 100c according to the second embodiment, various types of information such as the name (manufacturer's name) of the imaging device 100a (100c), status, existing position, user's name, fittings, battery level, travel distance, next maintenance date, and priority of maintenance may be displayed on the display 34 or 18, as shown in FIG. 14A, for example.

This can meet the various demands from parties relevant to the medication cart 1 (1A). In addition, since the statuses of a plurality of medication carts 1 (1A) can be recognized at a glance, it is possible to prevent only some of the medication carts 1 (1A) from wearing out by periodically rotating the medication carts 1 (1A), which can increase the efficiency of operations.

In addition, a facility manager wishes to consider the opportunity of equipment investment for the medication cart 1 (1A), and a supervisor of users wishes to consider allocation of shifts of the respective users. In addition, a service representative of a manufacturer of the imaging device 100a (100c) and the facility manager wish to make it easy to consider the frequency of maintenance of the medication cart 1 (1A), and the manufacturer wishes to obtain market information for product development. That is, parties relevant to the medication cart 1 (1A) wish to visualize the operating rate of the medication cart 1 (1A).

Thus, in the operation management system 100b according to the above-described first embodiment or the imaging device 100c according to the second embodiment, time information (date and day of the week) may be added to positional information acquired from the medication cart 1 (1A), as shown in FIG. 14B, for example.

This enables the differences in operation (imaging duration, movement duration, route, and the like) between dates to be understood, and plans for replacement, maintenance, and the like of equipment to be easily made.

Although embodiments of the present invention have been described and illustrated in detail, the disclosed embodiments are made for purposes of illustration and example only and not limitation. The scope of the present invention should be interpreted by terms of the appended claims.

1. An operation management system for managing a mobile radiation generating device having a radiation generator capable of generating radiation, the mobile radiation generating device being configured to be movable, the operation management system comprising:
a hardware processor that
repeatedly acquires positional information including a position at which the mobile radiation generating device is present and a time when the mobile radiation generating device is present at the position,
sequentially records the positional information as repeatedly acquired in a memory,
generates operation information related to a past operation of the mobile radiation generating device on a basis of a plurality of pieces of positional information as recorded, and
causes a display to display the operation information as generated.

2. The operation management system according to claim 1, wherein the hardware processor
further acquires information about at least any of imaging information related to details of imaging through use of the mobile radiation generating device, operator information related to an operator who operates the mobile radiation generating device, subject information related to a subject to be an imaging target in imaging through use of the mobile radiation generating device, and facility information related to a facility in which the mobile radiation generating device is included, and
generates the operation information on a basis of the further acquired information among the imaging information, the operator information, the subject information, and the facility information, as well as the plurality of pieces of positional information.

3. The operation management system according to claim 2, wherein the hardware processor
generates path information related to a movement path when the mobile radiation generating device moves within the facility on a basis of the plurality of pieces of positional information as recorded and the facility information, and
causes a plan view of an inside of the facility to be displayed on a basis of the facility information, and causes the movement path of the mobile radiation generating device to be displayed in a superimposed manner on the plan view on a basis of the path information as generated.

4. The operation management system according to claim 3, wherein
branch point information related to a branch point of a passage along which the mobile radiation generating device can move within the facility is included in the facility information, and
the hardware processor divides the path information as generated using the branch point indicated by the branch point information as a boundary to respectively generate partial path information related to partial paths corresponding to respective sections obtained by separating the passage with the branch point.

5. The operation management system according to claim 4, wherein the hardware processor generates section data including a movement duration required for the mobile radiation generating device to move through a corresponding section on a basis of the partial path information as generated.

6. The operation management system according to claim 5, wherein the hardware processor extracts partial path information corresponding to section data that achieves the shortest movement duration as shortest partial path information from a plurality of pieces of partial path information corresponding to an identical section, and combines pieces of shortest partial path information as extracted corresponding to respective sections to generate shortest path information related to a movement path that enables the shortest movement to an imaging place.

7. The operation management system according to claim 5, wherein the hardware processor changes a display mode of a section corresponding to a displayed movement path in accordance with an extent of a movement duration included in the section data.

8. The operation management system according to claim 5, wherein
waiting place information related to a waiting place of the mobile radiation generating device when not being used is included in the facility information, and
the hardware processor
generates shortest waiting place information related to a waiting place achieved the shortest movement duration to an imaging place at which imaging has been performed on a basis of the imaging information, the waiting place information, and a plurality of pieces of the section data, and
displays a position of the waiting place that achieves the shortest movement to the imaging place on the plan view on a basis of the shortest waiting place information.

9. The operation management system according to claim 5, wherein the hardware processor calculates an average value of respective movement durations on a basis of a plurality of pieces of the partial path information corresponding to an identical section.

10. The operation management system according to claim 2, wherein the hardware processor generates an imaging duration required for imaging on a basis of a plurality of pieces of the positional information and the imaging information as acquired.

11. A mobile radiation generating device, comprising:
a radiation generator capable of generating radiation; and
a hardware processor that
acquires positional information including a position at which the mobile radiation generating device is present and a time when the mobile radiation generating device is present at the position,
records the positional information as acquired in a memory,
generates operation information related to a past operation of the mobile radiation generating device on a basis of the positional information as recorded, and
causes a display to display the operation information as generated, wherein
the mobile radiation generating device is configured to be movable.

12. A mobile radiographic imaging system, comprising:
the operation management system as defined in claim 1;
a mobile radiation generating device having a radiation generator capable of generating radiation and being configured to be movable; and
a radiation detecting device that generates a radiographic image in accordance with received radiation.
